(19) United States
(12) Patent Application Publication
(10) Pub. No.: US 2026/0151086 A1
(43) Pub. Date: Jun. 4, 2026

(54) CONTROL SYSTEM AND USER INTERFACE FOR PROSTHETIC DEVICES

(71) Applicant: Infinite Biomedical Technologies, LLC, Baltimore, MD (US)

(72) Inventor: Rahul Kaliki, Baltimore, MD (US)

(21) Appl. No.: 19/402,972

(22) Filed: Nov. 26, 2025

Related U.S. Application Data
(60) Provisional application No. 63/726,517, filed on Nov. 30, 2024.

(51) Int. Cl. A61B 5/00; A61B 5/11; A61B 5/389

(52) U.S. Cl. CPC A61B 5/6811; A61B 5/1123; A61B 5/389; A61B 5/4851; A61B 5/743; A61B 5/7435; A61B 5/7475

(57) ABSTRACT

Embodiments are directed to prosthetic systems that include an electronic device configured to receive first sensor data based on signals detected by a first sensor and second sensor data based on signals detected by a second sensor. The electronic device can determine a resultant vector using the first sensor data and the second sensor data, display a user interface comprising a multi-dimensional plot corresponding to a posture map defining an association between one or more boundary regions and one or more posture definitions used to control movement of a prosthetic device. The multi-dimensional plot can include a first visual object defining a first boundary region corresponding to a first posture definition and a second visual object defining a second boundary region corresponding to a second posture definition. The electronic device can receive a user input to the multi-dimensional plot and update the posture map based on the user input.

### CROSS-REFERENCE TO RELATED APPLICATION

This application is a nonprovisional of, and claims the benefit under 35 U.S.C. §119(e) of, U.S. Provisional Patent Application No. 63/726,517 filed Nov. 30, 2024, the contents of which are incorporated herein by reference as if fully disclosed herein.

### FIELD

The described embodiments relate generally to control systems for user-worn electromechanical prosthetic devices and in particular to control systems for setting operating parameters of prosthetic devices.

### BACKGROUND

Prosthetic devices can be used to restore partial or complete limb function. A prosthetic device can use sensors to detect muscle, nerve or other biological signals and interpret those signals as pose or movement instructions that, in turn, can be used to drive one or more electromechanical actuators of the prosthetic device. A prosthetic device may be programmed to correlate specific signal patterns as an instruction to perform a specific pose or movement. This can be repeated for multiple poses or movements.

### BRIEF DESCRIPTION OF THE DRAWINGS

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

In particular, the disclosure will be readily understood by the following detailed description in conjunction with the accompanying drawings, wherein like reference numerals designate like structural elements, and in which:

FIG. 1 depicts an example prosthetic device that can be used with the control signal user interface as described herein;

FIG. 2 depicts an example system diagram of a control system of a prosthetic device as described herein;

FIG. 3 depicts an example process for creating and updating a posture map that is used to control operation of a prosthetic device;

FIGS. 4A-4B show an example user interface for creating and updating a posture map, as described herein;

FIGS. 5A-5C show an example user interface for assigning posture definitions of a posture map, as described herein;

FIG. 6 shows an example user interface for creating and updating a posture map, as described herein;

FIGS. 7A-7C show an example user interface for defining boundary regions of a posture map, as described herein;

FIGS. 8A-8B show an example user interface for defining boundary wall objects of a posture map, as described herein;

FIGS. 9A-9B show an example user interface for defining secondary correlation of a posture map, as described herein;

FIGS. 10A-10B show an example user interface for adding additional boundary regions to a posture map, as described herein;

FIG. 11 depicts an example system diagram of an electronic device that may perform operations as described herein.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Embodiments described herein are directed to a control signal user interface that can be used to view and modify parameters defined by a posture map. As used herein, the term “posture map” includes computer readable instructions or any suitable stored data format that defines correlations between sensed signals and one or more movement classifications, which can be used to control operation of a prosthetic device. For example, a posture map can define correlations between sensed user signals (e.g., myoelectric signals) and one or more movements or poses that can be performed by a prosthetic device. A pose may correspond to a defined relational position of a prosthetic device, and a movement may include motions of the prosthetic device to reach a pose and/or movements that perform one or more actions. For example, in the context of a prosthetic hand, a pose may include positions of the hand that correspond to a pointing of a finger, an open hand position, a closed hand position, and so on. A movement may be the motion that prosthetic elements (e.g., prosthetic fingers) perform to move between different poses and/or movements used to perform an action such as grabbing an object, applying pressure to hold an object and so on.

Additionally or alternatively, a posture map can define correlations between sensed user signals and other functions that relate to control of a prosthetic device. For example, the posture map may define correlations between sensed user signals and a play/pause function for a prosthetic device; a function that cycles through one or more programmed poses or movements for the prosthetic device; provide the ability to switch between different posture maps (e.g., maps having different posture definitions, and/or other functions related to control of the prosthetic device.

The systems and methods described herein leverage a posture map to identify an intended user (which also may be referred to as a “patient” or “wearer” or “operator” of a prosthetic device) pose/movement from detected volitional user signals, which may include myoelectric signals, sonomyographic signals, implantable neural signals, and/or suitable biological signals. User signals can be detected using sensors and the detected user signals can be analyzed to determine a resultant vector. The posture map can include different regions which each correspond to a different pose or movement. The resultant vector, determined from the user signals, can be correlated to a region on a posture map to determine an intended user pose or movement.

In some cases, the posture map defines a feature space using one or more component dimensions each corresponding to a different sensor. Different regions of the feature space can be associated with different posture definitions and different posture definitions can be used to control operation of a prosthetic device (e.g., cause the prosthetic device to perform a specific pose and/or movement). The resultant vector can be correlated to a location of the feature space, defined by the posture map. The location of the resultant vector within the posture map can be used to determine the intended user movement. For example, the location of the feature vector corresponding to a particular region associated with a particular posture definition can be used to control the prosthetic device to perform an operation (e.g., pose and/or movement(s)) for the particular posture definition. Collectively, a pose and/or movement may be referred to as an “operation” that can be performed by a prosthetic device.

In some cases, the posture map can be defined as a vector space and each direction component of the vector space can correspond to a different sensor. The feature vector can be determined as a vector combination (e.g., summation) of the signals (e.g., signal magnitude) and direction associated with each sensor. For example, in a two-dimensional posture map, the resultant vector can be a vector defined by combination of a magnitude of a first sensor signal having a first component direction and a magnitude of a second sensor signal having a second component direction. In some cases, the first component direction and second component direction can be defined as orthogonal component directions, however, the component directions can have other relationships. Additionally or alternatively, additional sensors may represent additional component directions (e.g., a third orthogonal component direction) and/or negative/opposite directions (e.g., first component direction extends in a first direction from an axis and second component direction extends in the opposite direction from the axis).

The sensors can detect the user signals over a period of time, which can be used to determine one or more intended operations. The system can dynamically update the feature vector based on changes of the detected user signals. These changes can cause the feature vector to transition between different regions of the posture map. Each region (also referred to herein as a “boundary region” or “boundary regions”) can be associated with a different posture definition, and the system can cause the prosthetic device to perform a particular operation(s) based on the current correlation between the feature vector and particular region defined by the posture map. Accordingly, as user signals are detected by one or more sensors, a changing correlation between the feature vector and the posture map (e.g., changes in position of the feature vector with respect to the posture map) can be used to determine an intended posture definition and/or changes in the intended posture definition and cause the prosthetic device to perform the intended operation.

The systems and methods described herein can include a control signal user interface that is configured to display a visual representation of a posture map and allow a user to change parameters of the posture map. For example, user inputs to the control signal user interface can be used to define boundary regions, a posture definition(s) associated with a boundary region, and/or other parameters that are used to control operation of a prosthetic device. The control signal user interface can be displayed as a multi-dimensional plot that includes visual objects that represent boundary regions. Each boundary region can be defined as an area within the feature space defined by the posture map. Each boundary region can be assigned and/or correlated to a different posture definition(s). This can allow a user to visualize how the posture map correlates different regions in the component space to different posture definitions.

The plot can be a multi-dimensional plot that displays the feature space according to component dimensions corresponding to different sensors. For example, each sensor that is used to measure user signals can define a different component dimension. A two-dimensional plot may include two orthogonal axes, where positive values along a first axis correspond to a first sensor, negative values along the first axis correspond to a second sensor, positive values along a second axis correspond to a third sensor, and negative values along the second axis correspond to a fourth sensor. In other cases, the plot can be displayed using other dimensional schemes such as a one-dimensional plot, a three-dimensional plot and so on, and/or using non-orthogonal axes.

The control signal user interface can display a user's current resultant vector location that is determined in real-time (or near-real time) from sensors that are contacting a user. The control signal user interface can display the user's current data along with one or more boundary regions, which can allow a user to see how a current muscle contraction would correlate to the one or more boundary regions. For example, as a user performs a current contraction, the system can measure the contraction pattern (e.g., using electromyography (EMG) sensors) and determine a current resultant vector for the contraction. The resultant vector may be time dependent and vary as the muscle activity of the user varies during the user's contraction. The current resultant vector can be displayed on the multi-dimensional plot with respect to the defined boundary regions. For example, if the resultant vector is displayed on a two-dimensional plot according to two component dimensions, the resultant vector can be displayed using two component dimensions on the two-dimensional plot.

In some cases, the resultant vector can be dynamically displayed using a moving cursor and/or a trace that is based on the time varying changes of the measured sensor data over a duration of muscle contraction. Accordingly, the control signal user interface can display how the resultant vector from a user's contraction changes over time with respect to the posture map and with respect to the boundary regions. Put another way, a user can perform a contraction and the control signal user interface can display the resultant vector associated with the contraction in real-time so that a user can see how their contraction relates to the defined boundary regions associated with different posture definitions (which may also be referred to herein as “movement classifications”). In this manner, a user may be able to practice particular contractions and the control signal user interface can display how those contractions are being classified in real time. In some cases, as a user performs a contraction, the control signal viewer may determine that the user's contraction would be classified as a particular movement (e.g., within a boundary region corresponding to a particular posture definition). In this way, a user can visualize if a current contraction is being classified as the intended movement.

In some cases, the system and methods described herein can be configured to utilize multiple posture maps and include the ability to switch between different ones of the multiple posture maps. For example, a first posture map can include a first set of posture definitions which correlate sensed signals to intended pose(s)/movement(s). The system may have a second posture map that includes alternative posture definitions having a second set of posture definitions with different correlations between sensed signals and intended pose(s)/movement(s). In some cases, a second posture map may have different pose(s)/movement(s) defined for similar sensed signals, which may provide alternative or additional movement capabilities. For example, using a first posture map, a user may be able to perform a first signal activation to perform a first pose/movement. The user may switch to a second posture map, and the same or similar signal activation can be used to cause the prosthetic device to perform a different pose/movement, and/or other function. In some cases, the user may manually switch between posture maps. In other cases, a specific signal activation (e.g., myoelectric activation pattern) can be correlated with a switch function and used to switch between different posture maps. For example, a user may perform a specific signal activation to switch between posture maps. In other cases, the system may be configured to switch between different posture maps based on one or more defined conditions. For example, in response to performing a first pose/movement, the system can be configured to switch to a different posture map, which may provide additional functionality (e.g., poses or movements). If the first pose of movement is associated with a point movement/pose, the second posture map may be associated with controls for the point movement or pose, for example finer controls for manipulating the point pose.

More generally and broadly, the systems and methods described herein relate to adaptive posture definitions that can be changed in response to determining an intended user movement and/or pose for a prosthetic device. The viewing techniques may allow a user to visualize how the control system is classifying muscle contractions and allow a user to change the currently defined boundary regions of a posture map to improve the performance of a user's control over a prosthetic device. In addition, the systems and methods described herein may result in reduced rejection rates and reduced instances of discontinued prosthetic use.

For simplicity of description, the embodiments described herein reference externally worn prosthetics (which may also be referred to as exoprostheses), but it may be appreciated that this is merely one example of a prosthetic that can leverage the systems and methods described herein. For example, some embodiments described herein can apply equivalently to endoprostheses, implanted biometric devices, non-prosthetic prosthetic devices, and so on. Further, for simplicity of description, many embodiments that follow reference a prosthetic configured for use by a user with an upper limb amputation, such as a transhumeral amputation or a transradial amputation. However, it may be appreciated that these are merely examples and other prosthetic devices can leverage the systems and methods described herein; these examples are not exhaustive. For example, the embodiments described herein can be used with orthoses, exoskeletons and/or other electromechanical systems.

Generally and broadly, as noted above, a user may provide instructions to a prosthetic system device (e.g., a myoelectric prosthetic device) by activating skeletal muscles and/or nerves in a residual limb and/or surrounding areas or other locations (e.g., chest, back, and so on). Activating skeletal muscle and/or nerves can generate electrical signals that can be detected by one or more electrodes (also referred to herein as “sensors,” “myoelectric sensors” or “EMG sensors”) positioned on the user's skin and/or implanted within the user's musculature. Additionally or alternatively, activated skeletal muscle and/or nerves can generate muscle activity (e.g., spatial movement) that can be detected using ultrasonic techniques (also referred to herein as “sensors,” or “sonomyographic sensors”) positioned on a user and/or implanted at least partially within the user's body. A user can generate different patterns of volitional motor control signals (e.g., myoelectric signals, sonomyography signals, neural signals and/or other suitable biological signals) that can be associated to specific movements or poses of the prosthetic.

The examples described herein are presented in the context of using EMG signals to create and/or calibrate posture maps that are used to identify intended user movement. However, other types of user input can be used to create, calibrate and/or otherwise develop classification models that are used to identify intended user movement. For example, sonomyography signals and/or neural signals can be used as an alternative to or in addition to EMG signals. The sonomyography signals and/or neural signals may be obtained by one or more sensors contacting the user (and/or implanted within the user) and the signals can be used to classify user movements using the techniques described herein.

For example, when a user intends their prosthetic to perform a specific action, the user can generate a unique electric pattern which can be sampled by a set or array of electrodes/sensors. Output from the sensors can be used to determine a resultant vector. The resultant vector can be correlated to a boundary region of a posture map that is associated with a posture definition/movement classification. The posture definition (movement classification) can thereafter be used by an electromechanical control system of the prosthetic to change an angular and/or linear position of one or more electromechanical actuators of the prosthetic, thereby moving the prosthetic and/or changing a pose thereof or causing a defined movement.

The control system user interface may also be used as a training application without the need of a prosthetic device. For example, the system can include an array of sensors that are attached to the user's limb and the signals obtained from the sensors can be used to perform various movement calibrations/definitions as described herein. Additionally, the control system viewer can display the boundary regions associated with a posture definition, along with real-time contraction data for a user, which may allow a user to practice contractions while receiving real-time visual feedback. The control system user interface can allow these operations to be done without needing to actuate a prosthetic device, which may allow a user to train and/or program a posture map prior to receiving or otherwise needing to wear the actual prosthetic device.

In some cases, the control system user interface can be implemented as a virtual reality (VR), augmented reality (AR), and/or mixed reality (MR) system, which may be generally referred to as a “virtual reality or VR” system. The VR system may generate a VR environment using a head mounted display. The VR environment may include simulated environments for calibrating a classification model, viewing posture maps and/or updating the posture maps as described herein. For example, the VR system may generate a VR environment that displays a multi-dimensional plot of a posture map along with a real-time view of the current resultant vector determined from a user contraction. The VR environment may display a relative positioning of the resultant vector within multi-dimensional space and within the context of the user's current environment, which may help a user view or engage with how the control system is mapping contractions to a posture map.

In some cases, the VR system may display feedback or other visual cues to a user that indicate EMG patterns of a contraction and/or how particular contractions are being classified. For example, the VR system may overlay image data onto the array of sensors that indicates how much EMG activity each sensors is currently detecting. Accordingly, the user may receive visual cues at the sensor site that indicates a current contraction pattern. Additional or alternatively, the posture map can be concurrently displayed in the VR environment, which can create a direct visual correlation between a current contraction pattern and how that contraction is being correlated with the posture map, which is represented in the component space.

In other cases, the VR system can be used to coach and/or provide suggestions to help a user perform contractions that result in a desired movement classification. For example, the system may display a target contraction pattern over the sensor array that would result in a resultant vector and/or different posture map boundary regions that would reduce misclassifications. Accordingly, a user may try to emulate the displayed contraction pattern. These are just some examples of how aspects of the present disclosure can be integrated into a VR environment.

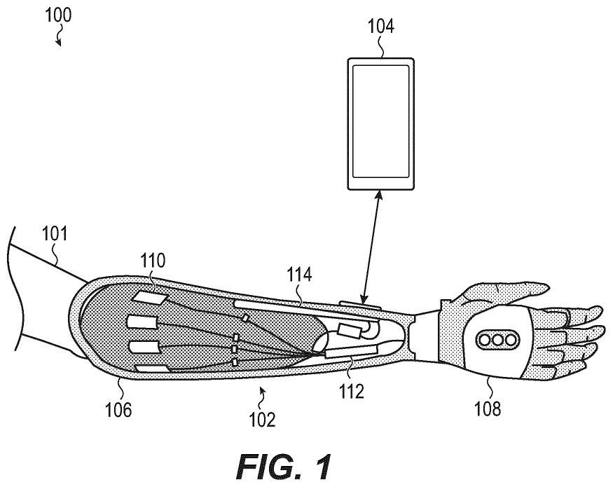

These and other embodiments are discussed below with reference to FIGS. 1-11. However, those skilled in the art will readily appreciate that the detailed description given herein with respect to these Figures is for explanatory purposes only and should not be construed as limiting.

FIG. 1 shows an example of a prosthetic system 100 (which may also be referred herein as a “prosthetic system”) including a prosthetic device 102, and an electronic device 104 that displays a user interface generated by the signal control viewer. The prosthetic device 102 can include a frame 106 that fits over a residual limb 101 of a user. The frame 106 can couple to an electromechanical device 108, such as a prosthetic hand. In some cases, the electromechanical device 108 can include components that control movement and/or other functions of the device. For example, the electromechanical device 108 can include motors, sensors, a control system that controls movement of the device, and so on. In some cases, the electromechanical device 108 can include an independent power source. In other cases, the electromechanical device 108 can use a power source (e.g., power source 114) positioned on the frame 106.

The prosthetic device 102 can include one or more sensors 110 (one of which is labeled for clarity), a control system 112, and a power source 114. The control system 112 can exchange control commands and/or other data with the electromechanical device 108. For example, in cases where the electromechanical device 108 is a prosthetic hand, the control system 112 may send commends to the prosthetic hand that instruct the prosthetic hands to perform a specific movement and/or pose, such as opening the fingers of the hand. The prosthetic hand may receive the command from the control system 112 and one or more onboard processors can cause the hand to perform the requested operation(s).

The sensors 110 can be configured to detect volitional motor control signals (e.g., myoelectric signals, sonomyography signals, neural signals, and/or other suitable biological signals) at the user's limb 101 and/or at other locations of the user. The sensors 110 can include multiple electrodes that couple to the frame 106 and contact the user's residual limb 101 when the prosthetic device 102 is worn by the user. Additionally or alternatively, the sensors 110 can include ultrasonic sensor(s) that are coupled to the frame 106 and contact the user's residual limb 101 when the prosthetic device is worn by the user. The sensors 110 can be communicably coupled to the control system 112.

The power source 114 can be coupled to the frame 106, and in some cases, may be positioned on an inside portion of the frame. The power source 114 may be a flexible power source that can conform to an interior surface of the frame 106 and/or a portion of the user's residual limb 101. The power source 114 can include a battery or other suitable power source as described herein and may be removed and/or recharged.

In some cases, the frame 106 can include a coupling mechanism that allows different electromechanical devices 108 to be removably coupled to the frame 106. Additionally, the coupling mechanism can allow other types of electromechanical devices to be coupled to the frame and/or the frame 106 can include different types of coupling mechanisms that couple with other electromechanical devices. Accordingly, the frame 106, the sensors 110, the control system 112, and the power source 114 may form a sub-prosthetic that can interface with different types of actuating protheses. This sub-prothesis may primarily function to detect a user's volitional motor control signals (e.g., myoelectric signals, sonomyography signals, and/or other suitable biological signals), process those signals to determine an intended movement, and send control commands to an electromechanical device 108 for performing the intended movement.

The electronic device 104 can be any suitable electronic device that includes a display and is communicably coupled with the prosthetic device 102 including a smartphone, tablet, smartwatch, computer, or other electronic device. The electronic device 104 can be communicably coupled to the control system 112 via any suitable wired or wireless transmission protocol. In yet other cases, the electronic device 104 can be integrated with the frame 106.

The electronic device 104 can be configured to output information to the user and receive inputs from the user. In some cases, the electronic device 104 can include a touch-sensitive display, audio, haptic, and/or other output mechanism. The electronic device 104 may also receive a variety of input types from a user such as touch inputs (e.g., to the touch-sensitive display), speech inputs such as voice commands, and/or other suitable input types. The electronic device 104 can transmit indications of received user inputs to the control system 112. Accordingly, a user may use the electronic device 104 to visualize data or other control signals generated by the prosthetic device 102 and/or to provide feedback to the prosthetic device 102.

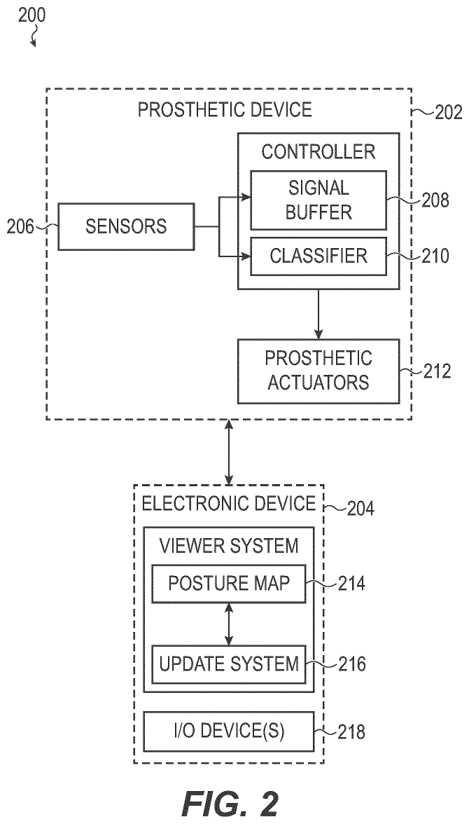

FIG. 2 shows an example process diagram 200 for control of a prosthetic device. The process diagram shows example operational elements of a control system that can be used to control a prosthetic device 202, and/or interface with an electronic device 204, and communications between these devices. The prosthetic device 202 can be an example of the prosthetic devices described herein and the electronic device 204 can be an example of the electronic devices described herein.

The prosthetic device 202 can include one or more sensors 206 (e.g., electrodes, ultrasonic sensors, neural sensors, and/or other suitable sensors) which can be examples of the volitional motor control sensors described herein (e.g., sensors 110). For example, the sensors 206 may sense myoelectric signals (and/or other muscle activity) of the user's limb and output data indicative of the measured myoelectric activity, sonomyographic activity, and/or other sensed signals to a signal buffer 208, which may store the data (e.g., myoelectric data, sonomyography data) for a period of time. In some cases, the data may be stored until the classifier 210 has processed the data, and/or the prosthetic actuators 212 have performed a movement. The sensors 206 may also output the sensed user data to the classifier 210.

The classifier 210 can use a posture map, and input detected sensor signal data as inputs. The classifier 210 can determine a resultant vector based on the sensor signal(s) and determine a correlation of the resultant vector to the posture map. The classifier 210 can output one or more determined posture definitions (movement classifications) based on the correlation between the resultant vector and the posture map. The posture definition can be used to cause the prosthetic device to perform the corresponding movement or pose. For example, the posture definition may be transmitted to a prosthetic device (or used to select a command) that is used to control operation of the prosthetic device. Accordingly, the control system can output control signals that cause a prosthetic device to perform a specific movement based on the outputs from the classifier 210.

The output of the classifier 210 can be used to send control commands to the prosthetic actuators 212 (e.g., in the form of a posture definition or other suitable command). The prosthetic actuators 212 can include the components in a prothesis that generate motion such as hand motion(s) instructed by control commands from the controller. In some cases, the control commands can cause the prosthetic to perform operations including a movement such as opening the hand, closing the hand, or moving the hand to a specific pose/position. In other cases, the control commands can include instructions for performing more complex operations such as a sequence of movements and/or poses. For example, the control commands can instruct a sequence of movements to pick up an object and cause the prosthetic actuators 212 to perform a sequence of movements such as first opening the hand so that it can be positioned around an object and then closing the hand to grip the object.

In some cases, the prosthetic actuators 212 can include one or more sensors, which may be used to control the timing of the movements. In the sequence of opening a hand followed by closing of a hand to pick up an object, the sensors may be used to determine when the hand is ready to be closed. For example, the prosthetic actuators 212 may perform the first motion of opening the hand and then wait for feedback from one or more sensors indicating that the hand is positioned around an object. In response to the feedback, the prosthetic actuators 212 may perform the second movement of closing the hand. The electronic device 204 can be sent an indication of the movements performed by the prosthetic actuators 212. In some cases, the controller may send indications of the control commands to the electronic device 204.

In some cases, the prosthetic device 202 can communicate with the electronic device 204. For example, the classifier 210 outputs and/or prosthetic actuators 212 control signals may send feature data and/or movement control data to the electronic device 204, which may be used to by the viewer system to display data associated with sensed/detected data. The electronic device 204 can implement a viewer system (also referred to herein as a “control signal viewer”), which can display a visual representation of a posture map and/or a resultant vector determined from the sensed data.

The control signal viewer system (or “viewer system”) can include a posture map 214, which can receive data from the prosthetic device including control data that is used to operate the prosthetic actuators 212. The viewer system can process the received data and generate a user interface that displays the sensor data on the electronic device 204, as described herein. The viewer system can provide control that allows a user to select how and/or what data is displayed in the user interface. For example, the viewer system may allow a user to view a specific posture map. The viewer system may include controls for manipulating a view perspective of the data and so on, as described herein. The update system 216 can allow a user to obtain additional data (e.g., EMG data, sonomyography data) that can be incorporated into the classifier 210. The electronic device 204 can include an I/O device(s) 218 as described herein, which can be used to output information to the user and receive feedback from the user.

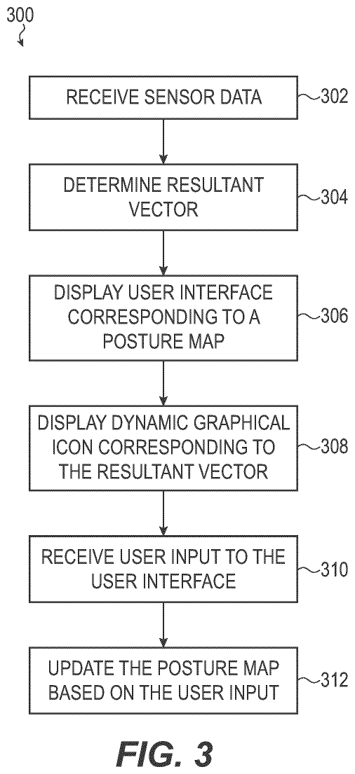

FIG. 3 depicts an example process 300 for creating and updating a posture map that is used to control operation of a prosthetic device. The process 300 can be performed by the systems and devices described herein such as the prosthetic system 100 and/or prosthetic system 1100.

At operation 302, the process 300 can include receiving sensor data from one or more sensors that are configured to measure user signals. For example, the sensor data can be created from and/or by the sensors described herein including sensors 110, sensors 206, and/or signal buffer 208. The sensor data can include raw sensor signals, filtered, conditioned, digitized, and/or sensor signals that have undergone one or more processing steps.

The sensors can include a first sensor configured to detect first signals at a first location of the user and a second sensor configured to detect second signals at a second location of the user. The system can include additional and/or fewer sensors. The sensors can include sensors that are configured to detect signals that are related to volitional motor control of a user. For example, the sensors may include myoelectric sensors, as described herein, and are configured to detect user signals (e.g., electrical signals associated with muscle contraction) that are associated with control of a portion of the user's body such as a hand and/or a limb. In other cases, the sensors can include sonomyography sensors which may detect movement of muscle, neural sensors which may detect neural signals, other sensor types and/or combinations thereof.

At operation 304, the process 300 can include determining a resultant vector using the detected sensor signals. The resultant vector can be determined based on a combination of the sensor signals and represent the combination in a posture map. The posture map may be defined by component dimensions. In some cases, each sensor signal can correspond to a component dimension and the resultant vector can be a vector combination of the sensor signals (e.g., a linear combination of sensor signals). For example, the electronic device can receive first sensor data based on the first myoelectric signals detected by the first sensor and receive second sensor data based on the second myoelectric signals detected by the second sensor. The first sensor data can correspond to a first component dimension and the second sensor data can correspond to a second component dimension. The resultant vector can be a linear combination of the first sensor data and the second sensor data and define a location within the posture map.

In other cases, the system can include additional sensors, which can define additional component dimensions in the posture map. For example, the posture map can be one-dimension, two-dimensions, three-dimensions, etc., and each dimension may correspond to one or more sensors. In some cases, a first sensor may correspond to “positive” values along a first component dimension and a second sensor may correspond to “negative” values along the first component dimension. In other cases, the first sensor and second sensor can correspond to different component dimensions. Accordingly, the resultant vector can include combinations of sensor signals represented in a single sensor component dimension or multiple component dimensions. The resultant vector can represent both a direction/location and magnitude of the combination of the sensor signals within the posture map.

At operation 306, the process 300 can include displaying a user interface corresponding to a posture map. For example, the user interface can include a multi-dimensional plot corresponding to a posture map represented by the component dimensions. The posture map can define an association between one or more boundary regions and one or more posture definitions, which are used to control movement of a prosthetic device, as described herein. In some cases, the multi-dimensional plot defines multiple boundary regions within the posture space. For example a first boundary region can define a specific are of the posture space and a second boundary region may define a second area of the posture space, different from the first area. In this regard, the posture space can be divided into multiple different boundary regions.

The multi-dimensional plot can include visual objects that provide a visual representation of the boundary regions within the posture space. For example, the multi-dimensional plot can include a first visual object defining a first boundary region and a second visual object defining a second boundary region. The visual objects can be interface elements that are configured to be modified within the user interface. The first boundary region/visual object can correspond to a first posture definition and the second boundary region/visual object can correspond to a second posture definition. In some cases, the user interface is configured to allow a user to assign a posture definition to each boundary region/visual object.

In some cases, the visual objects can be modified within the user interface to change a definition of the corresponding boundary region. The first visual object can define one or more selectable edges, which can be moved or otherwise modified to change the size and/or shape of the corresponding boundary region within the posture space. For example, the first visual object can include a first selectable edge corresponding to an activation threshold for the first posture definition, as described herein. The user interface is configured to allow user inputs to change a location of the first selectable edge within the multi-dimensional plot.

At operation 308, the process 300 can include displaying a dynamic graphical icon corresponding to a resultant vector. As sensor data is received, the resultant vector can be continually determined and/or updated and a location of the resultant vector can be displayed in the multi-dimensional plot. In some cases, the resultant vector can be displayed as a dynamic graphical icon on the multi-dimensional plot indicating a current correlation of the resultant vector with respect to the one or more boundary regions of the posture map. Accordingly, as sensor signals change (e.g., in response to changing contraction patterns or other volitional motor control changes), the resultant vector can be updated and the updated location can be displayed within the multi-dimensional plot. The resultant vector can be displayed in real-time or near real-time so that a user can view how the system is categorizing the detected signals - e.g., a current location of the resultant vector with respect to the defined boundary regions.

At operation 310, the process 300 can include receiving user inputs to the user interface. For example, the electronic device can receive a user input changing a location of at least a portion of a boundary region within the multi-dimensional plot. In response to a change to a boundary region, the electronic device can update the posture map.

At operation 312, the process 300 can include updating the posture map based on the user inputs. For example, the system can update the posture map based on the change to the location of the portion of the boundary region. Accordingly, the system can be configured to receive user inputs to the user interface and update the posture map based on the user inputs.

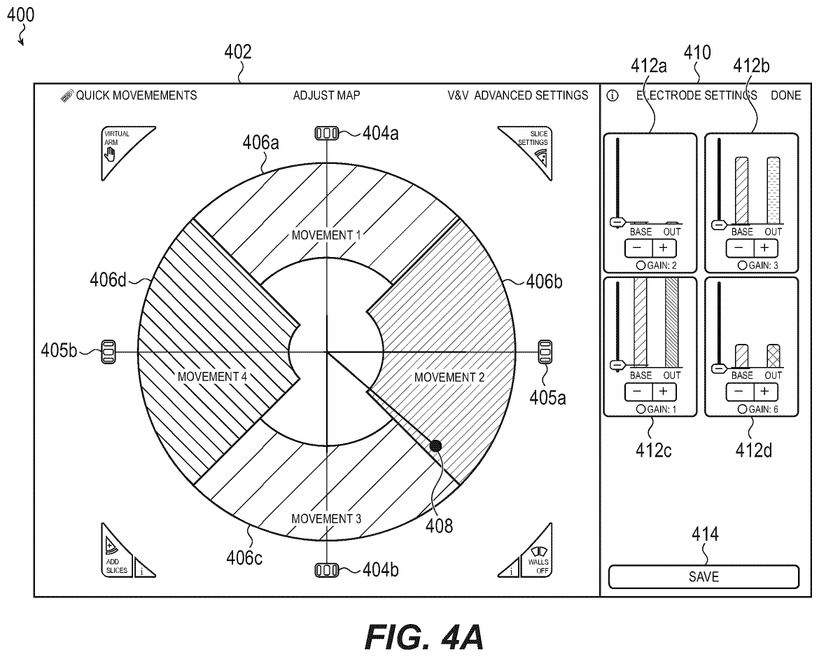

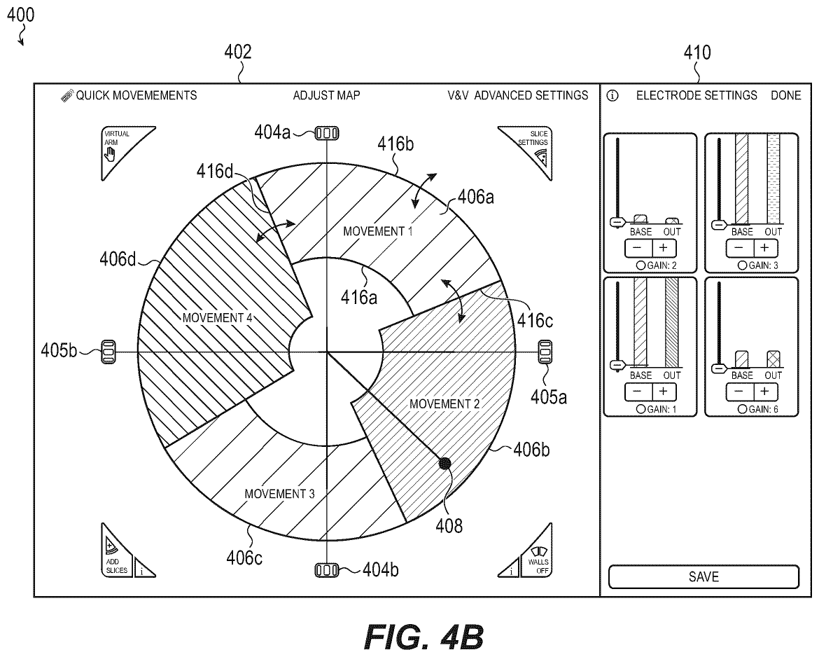

FIGS. 4A-4B show an example user interface 400 for creating and updating a posture map, as described herein. The user interface may also be referred to herein as a “control signal user interface.” The user interface 400 can display a visual representation of a posture map and be used to define, update and/or modify various parameters of a posture map, as described herein.

As shown in FIG. 4A, the user interface 400 can include a multi-dimensional plot 402 which provides a visual representation of the posture map using component dimensions 404a, 404b, 405a, 405b. Component dimension 404a, 405b may correspond to one or more sensors and be used to represent signals detected by the corresponding sensor(s). For example, a first component dimension 404a can correspond to signals measured by a first sensor (including the sensors described herein) and a second component dimension 404b can correspond to signals measured by a second sensor. In this regard signals measured by the first sensor have a direction and magnitude corresponding to the first component dimension 404a and signals measured by the second sensor have a direction and magnitude corresponding to the second component dimension 404b.

Component dimension 405a, 405b may correspond to one or more sensors and be used to represent signals detected by the corresponding sensor(s). For example, a third component dimension 405a can correspond to signals measured by a third sensor and a fourth component dimension 405b can correspond to signals measured by a fourth sensor. In this regards, signals measured by the third sensor have a direction and magnitude corresponding to the third component dimension 405a and signals measured by the fourth sensor have a direction and magnitude corresponding to the fourth component dimension 405b. The illustrated example provides an example of a posture map and prosthetic system having four sensors and four component dimensions. However, other numbers of sensors and/or corresponding component dimensions are possible such as systems having two sensors and two respective components dimensions, three sensors and three component dimensions, and so on.

The multi-dimensional plot 402 can include one or more boundary regions 406a, 406b, 406c, 406d (one or more of which may be referred to herein as “boundary region 406”) which are represented by visual objects in the user interface. Each boundary region 406 can include a defined portion of the posture map and be defined using the component dimensions. FIG. 4A illustrates an example multi-dimensional plot 402 having four boundary regions that include a first boundary region 406a (corresponding to “Movement 1”), a second boundary region 406b (corresponding to “Movement 2”), a third boundary region 406c (corresponding to “Movement 3”) and a fourth boundary region 406d (corresponding to “Movement 4”). Each boundary region may include an area of the posture map that is defined using combinations of the component dimensions.

Each boundary region can be assigned one or more posture definitions and otherwise be modified to define operations associated with a respective boundary region, as described herein. For example, the posture map can define an association between each boundary region and a respective posture definition, and the multi-dimensional plot can show a visual representation of these defined relationships. For example, the first boundary region 406a is represented using a first visual object showing the area of the posture map associated with the first boundary region 406a, the second boundary region 406b is represented using a second visual object showing the area of the posture map associated with the second boundary region 406b, and so on.

The multi-dimensional plot 402 can display a dynamic graphical icon 408 indicating a current correlation of a resultant vector with respect to the one or more boundary regions 406 of the posture map. A corresponding posture/movement can be performed in response to the dynamic graphical icon 408 being located within a boundary region, which is used to visually indicate that the resultant vector corresponds to a particular boundary region. The resultant vector can be determined from the detected sensor signals and represented as a combination of the component dimensions corresponding to each sensor signal. For example, the resultant vector can be a linear combination of the sensor signals. The dynamic graphical icon 408 can be a time-varying visual object that is updated to display changes in the detected sensor signals over time (e.g., during a muscle contraction). Accordingly, the location of the dynamic graphical icon 408 can change with respect to the multi-dimensional plot as the sensors signals change.

The user interface 400 can include additional menus and/or display areas that display information about the prosthetic system. For example, the user interface 400 can include a sensor settings display 410, which is configured to display sensor signal data corresponding to one or more of the sensors. For example, the sensor settings display 410 can include sensor data displays 412, which show currently detected signal values corresponding to each sensor. A first sensor data display 412a can show signal values corresponding to the first sensor, a second sensor data display 412b can show signal values corresponding to the second sensor, a third sensor data display 412c can show signal values corresponding to the third sensor, and a fourth sensor data display 412d can show signal values corresponding to the fourth sensor. In some cases, each sensor data display 412 can include controls for adjusting a respective sensor signal. For example, each sensor data display 412 can include gain controls, which can be used to define a gain associated with a respective sensor signal. Adjustments to these controls (e.g., gain of each signal) can be performed prior to determining the resultant vector from the signals. Accordingly, the adjustments to the sensor signal controls can be used to modify the determined resultant vector.

The user interface 400 can include a save option 414, which can be used to save the current settings and cause the corresponding posture map to be updated/saved based on the current definitions determined from the user interface 400. For example, a user may make changes to one or more boundary regions, a posture definition associated with a boundary region, gain controls, and so on, and the save option 414 can cause the posture map to be updated based on these changes.

FIG. 4A shows a first instance of the user interface 400 and FIG. 4B shows a second instance of the user interface 400 after changes to one or more of the boundary regions 406. In some cases, the position and/or area of one or more of the boundary regions 406 can be changed. For example, as a user attempts to perform a muscle contraction corresponding to an intended pose/movement, the system can determine and display a resultant vector 408 associated with that muscle contraction in the multi-dimensional plot 402. In some cases, the resultant vector, for the particular muscle contraction, may be positioned near a boundary between two of the boundary regions and/or may end up in different boundary regions for different repetitions of the same contraction. Accordingly, it may be desirable to change the location and/or area of one or more of the boundary regions so that the resultant vector, associated with that particular muscle contraction can repeatably end up in the desired boundary region 406.

For example, a user may desire for a particular muscle contraction to be associated with a particular posture definition thereby causing the prosthetic device to perform the intended operation. Accordingly, the user may desire the resultant vector 408 during the particular contraction to repeatably end up in the second boundary region 406b. Using the posture map shown in FIG. 4A, the resultant vector for one or more contractions may be positioned near the boundary between the second boundary region 406b and the third boundary region 406c and/or may sometimes be positioned in the second boundary region 406b and sometimes be positioned in the third boundary region 406c. Accordingly, in response to detecting the particular muscle contraction, the system would sometimes perform the operation (pose/movement) associated with the second boundary region 406b and sometimes perform the operation (pose/movement) associated with the third boundary region 406c. This may be undesirable, since the system would cause the prosthetic device to cause different movements in response to the same type of particular contraction.

A user of the system may update the location and/or area of the boundary regions 406 as shown in FIG. 4B. For example, a user may change a location of one or more borders of the second boundary region 406b and third boundary region 406c so that the resultant vector from the particular muscle contraction is repeatably located within the second boundary region 406b.

In some cases, each boundary region may be defined by a border that includes one or more side segments. For example, as illustrated in FIG. 4B the first boundary region 406a can include a border defined by a first side segment 416a, a second side segment 416b, a third side segment 416c and a fourth side segment 416d. This is one example and other boundary profiles/shapes are possible. Additionally, the features described with respect to the first boundary region 406a can be applied to the other boundary regions 406.

The visual object displayed in the user interface 400 and corresponding to the first boundary region 406a can allow a user to change a location of one or more of the side segments to change a corresponding parameter defined by the boundary region. For example, the first side segment 416a can correspond to an activation threshold that is met before performing the associated movement (e.g., “Movement 1”). The user interface 400 can allow a user to move the first side segment 416a closer to the origin thereby reducing the activation threshold or move the first side segment 416a further from the origin thereby increasing the activation threshold. The second side segment 416b can be associated with a deactivation threshold and be similarly manipulated closer to or further from the origin to change the deactivation threshold. In this example, the deactivation threshold may define parameters in which the system causes the corresponding pose/movement to stop being performed.

The third side segment 416c and the fourth side segment 416d can define side locations of the first boundary region 406a. In the illustrated example, the third side segment 416c and fourth side segment 416d can be moved in a radial direction to expand, contract or otherwise change the area and/or location of the first boundary region 406a. Changes to one or more of the boundary regions 406 can be saved, which can cause the corresponding posture map to be updated in accordance with the changes.

In some cases, the system can define paired transitions between movements. Paired transition (which may also be referred to as “paired activation”) allows the user to switch between two movements by entering a first movements boundary region (e.g., the first boundary region 406a for movement 1). This may enable rapid switching between paired movements.

When paired transitions are enabled, upon leaving an active boundary region with an assigned movement that is paired, a timer window can be initiated. During the timer window, the originating boundary region's movement label can be stored in a variable. As the cursor enters a boundary region while the pairing is enabled, the boundary regions assigned movement labels are checked against a pairing check value to see if the paring check values correspond to a paired movement definition. If, during the timer window, the cursor enters a boundary region associated with a paired movement definition (e.g., stored using a pairing check value), the paired movement definition for the boundary region is activated, and the paired movement is set as the output. The timer can be initiated in response to the resultant vector 408 leaving a boundary region via one of the borders (e.g., side segments 416a, 416b, 416c, 416d). For example, in response to the resultant vector 408 leaving the first boundary region 406a by passing the first side segment 416a a timer can be initiated. Accordingly, in this example, the timer can allow the previous movement definition (e.g., movement definition associated with the first region 406a) to be stored and/or remembered as the resultant vector 408 passes to a neutral region (e.g., across the inner boundary). In some cases, the timer window can be a defined value.

In some cases, the paired transitions can be performed in response to movement between some side segments and not others. For example, a paired transition can be performed in response to the resultant vector 408 leaving a first boundary region 406a by passing the first side segment 416a (e.g., the timer window is activated). However, a paired transition may not be performed in response to the resultant vector 408 leaving a first boundary region 406a by passing the third side segment 416c. In some cases, though transition from a first boundary region (e.g., the first boundary region 406a) to a second boundary region (e.g., the second boundary region 406b) via the resultant vector 408 crossing the third side-wall 416c can activate a timer window and/or cause a paired movement check for a paired movement associated with the second boundary region 406b.

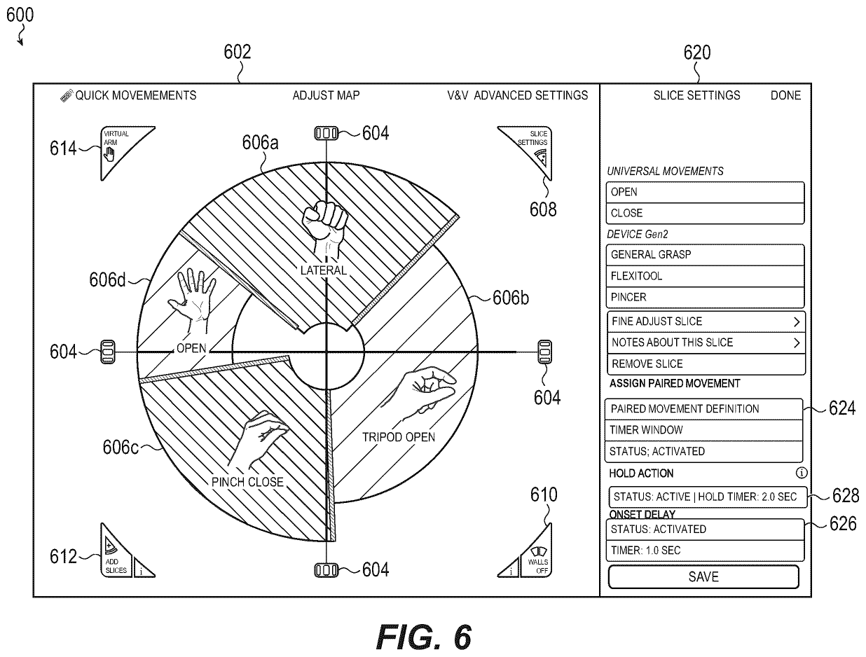

In some cases, paired movements may be antagonist commands, although the system may allow other types of movement definitions to be paired. For example, if a movement definition for a particular boundary region correspond to a hand close movement, the paired movement may be defined as a hand open movement. Other examples of primary movement and paired movement pairs include a grip movement and open movement; a palm up movement and palm down movement; a palm down movement and palm up movement; a wrist flexion movement and wrist extension movement; a wrist extension movement and a wrist flexion movement; an elbow flexion movement and an elbow extension movement; an elbow extension movement and an elbow flexion movement; and so on. FIG. 6 shows an example menu 626 for defining a onset delay for a boundary region (e.g., one or more of boundary regions 606a, 606b, 606c, 606d).

In some cases, the system can also define an onset delay function for one or more movements defined buy a boundary region. The onset delay feature can filter out inadvertent and unintentional primary activation of a boundary region 406a, 406b, 406c, 406d. Non-targeted boundary regions may be boundary regions through which the resultant vector 508 passes on its way to a targeted boundary region. The onset delay feature can define a time parameter for the resultant vector 408 to stay in the boundary region before the corresponding movement is activated. In some cases, the onset delay can be activated and deactivated and/or set to a defines value (e.g., using timer activation menu 626 shown in FIG. 6). The onset delay timer can be initiated once the vector enters a new boundary region either from a neutral/rest state or from a previously activated boundary region. The onset delay timer can be reset when the resultant vector enters a rest/neutral state and/or a enters a new/different boundary region. If, the onset timer is initiated from a rest/neutral state, the onset delay timer may not be reset when it travels across a side segment between two different boundary regions. The onset delay timer can be initiated when the resultant vector 408 leaves an activated boundary region and enters an adjacent boundary region without entering the rest/neutral region.

In some cases, the system can define a hold activation function. An action that is activated as a result of a hold activation function (or “hold action”) may be assigned to a primary action or secondary action of a given boundary region 406 as described herein. When a hold action is enabled, the hold action is activated when a user maintains the resultant vector 408 in a boundary region 406 for a specified hold time period after a primary and/or secondary movement/activation defined or a boundary region 406. A primary movement and/or secondary movement within a same boundary region 406 may each have an independent hold movement. Accordingly each boundary region 406 may be associated with multiple different actions including a primary posture definition, secondary posture definition, primary hold, and/or secondary hold. When a hold action has been triggered the system will activate a define hold action, which can may maintain a current posture, even if the resultant vector leaves the current boundary region 406. The hold action may be maintained until one or more conditions are met such as a defined hold time. FIG. 6 shows an example menu 628 for defining and/or activating a hold function for a boundary region (e.g., one or more of boundary regions 606a, 606b, 606c, 606d).

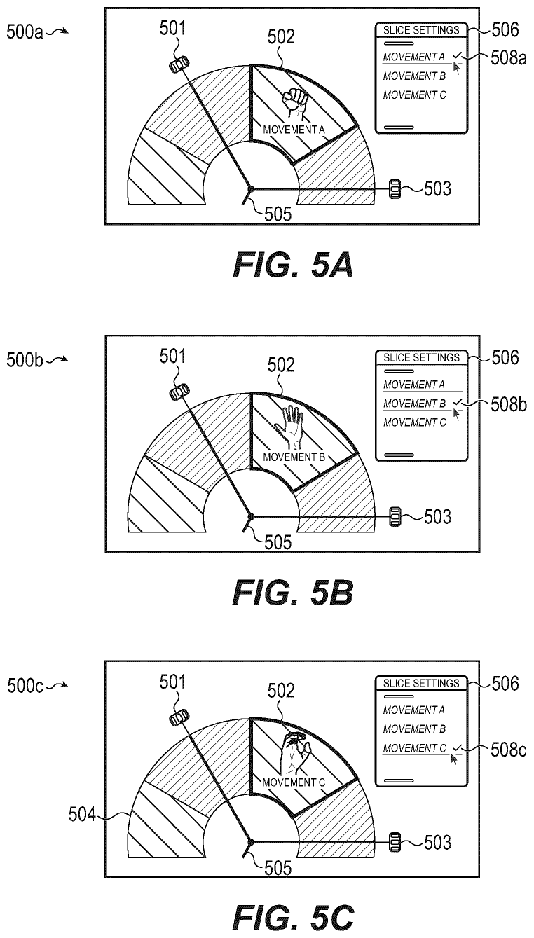

FIGS. 5A-5C show example user interfaces 500 for assigning a particular posture definition (and corresponding pose/movement) to a boundary region of a posture map, as described herein. The user interfaces 500 can be examples of the user interfaces described herein and include a multi-dimensional plot showing a visual representation of the posture map. In the examples shown in FIGS. 5A-5C the system includes three sensors and the posture map is defined using three component dimensions each corresponding to a different sensor. For example, each user interface 500 can include a first component dimension 501 corresponding to a first sensor, a second component dimension 503 corresponding to a second sensor and a third component dimension 505 corresponding to a third sensor.

FIG. 5A shows an example of assigning a first posture definition to a first boundary region 502 in a first instance of the user interface 500a. For example, in response to selection of the first boundary region 502, the user interface 500a can display a selection object 506 that includes multiple posture objects 508 each corresponding to a different posture definition. For example, a first posture object 508a corresponds to a first posture definition (“Movement A”), which causes the prosthetic system to perform a corresponding pose/movement. In response to detecting a selection of the first posture object 508a, the electronic device can assign the corresponding posture definition to the first boundary region 502. The posture map can be updated based on the assignment of the first selection object.

FIG. 5B shows an example of a selection of a second posture object 508b, which corresponds to a second posture definition (“Movement B”), and assignment of a second posture definition to the first boundary region 502. FIG. 5C shows an example of a selection of a third posture object 508c, which corresponds to a third posture definition (“Movement C”), and assignment of a third posture definition to the first boundary region 502. Accordingly, a prosthetic system can be configured with multiple posture definitions, and the user interfaces described herein (e.g., user interfaces 400 and 500) can be configured to allow a user to assign a particular posture definition to one or more of the boundary regions.

FIG. 6 shows an example user interface 600 for creating and updating a posture map, as described herein. The user interface 600 can be an example of the user interfaces described herein (e.g., user interfaces 400 and 500) and the posture map defined using the user interface 600 can be used to control operation of a prosthetic system as described herein.

The user interface 600 can include a multi-dimensional plot 602, which can be an example of the multi-dimensional plots described herein (e.g., multi-dimensional plot 402). In the example shown in FIG. 6, the prosthetic system includes four sensors each corresponding to a component dimensions 604, as described herein. The posture map includes four boundary regions, which are represented by visual objects 606 shown in the multi-dimensional plot 602. Each boundary region is assigned a different posture definition. For example the first visual object 606a displays an association of the first boundary region with a “lateral” posture definition, a second visual object 606b displays an association of the second boundary region with a “tripod open” posture definition, a third visual object 606c displays an association of the third boundary region with a “pinch close” posture definition, and a fourth visual object 606d displays an association of the fourth boundary region with a “open” posture definition.

The visual objects 606 represent an area and location of each region within the posture space defined by the posture map. The multi-dimensional plot 602 shows an example in which each boundary region has been adjusted to have differing boundary segments (e.g., activation threshold, and so on), which can be defined/set using the user interface 600 as described herein. For example, the user interface 600 may be configured to allow a user to select a boundary segment and drag it to a different location, enter a value for the selected boundary region, and/or other suitable process for setting parameters (e.g., location) of a boundary segment that defines the boundary region.

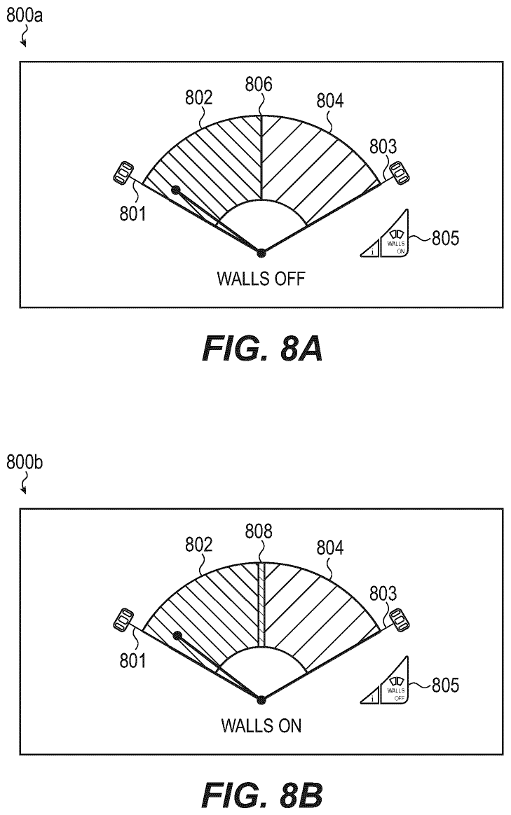

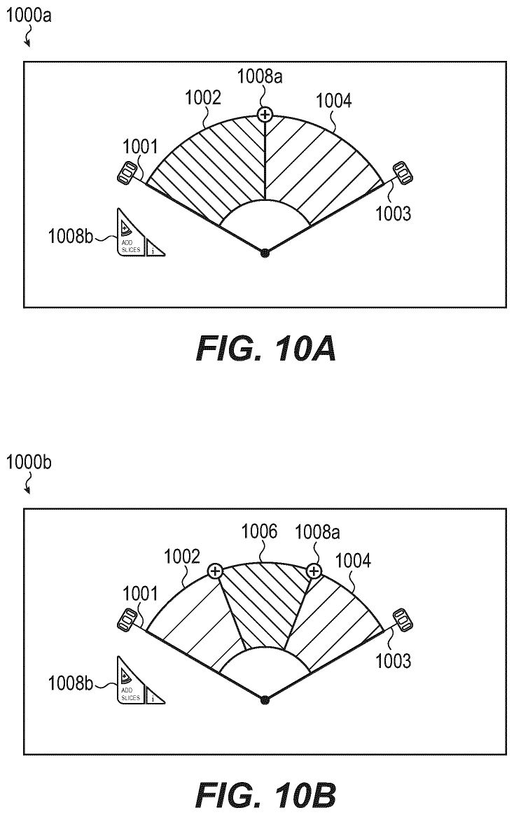

In some cases, the multi-dimensional plot 602 can include additional objects that are used to define parameters of the posture map. For example a slice object 608 can be used to control/activate slice settings of the multi-dimensional plot 602. Selecting the slice object 608 can allow a user to modify the boundary definitions to change a location of boundary segments, as described herein. In some cases, the system can be configured to only allow changes to the boundary definitions of the corresponding visual object 606, when the slice object 608 is selected. A wall object 610, may allow the addition of and/or setting parameters for a boundary wall, as described herein (e.g., with respect to FIGS. 8A and 8B). An add slices object 612, may allow the addition and/or removal of additional boundary regions, as described herein (e.g., with respect to FIGS. 10A-10B). A virtual arm slice 614 may cause the user interface 600 to display a visual representation of a prosthetic device (e.g., a prosthetic hand) and animate poses/movements based on classifications using the posture map defined by the multi-dimensional plot 602. Accordingly, a user may be able to try settings before uploading or otherwise configuring the hardware of the prosthetic device with the updated posture map.

In some cases, the user interface 600 can include additional menus and/or display areas that display information about the prosthetic system. For example, the user interface 600 can include a settings menu 620, which is configured to display additional posture map parameters and may allow for user inputs to define, change or otherwise modify these parameters. Settings defined in the settings menu 620 can be saved as part of the posture map. For example, the settings menu 620 can include a first option 622 for defining a posture definition, which may be an alternative or in addition to selecting a posture definition as described with respect to FIG. 5. For example, selecting the first option may cause the electronic device to display the selection object 506. The settings menu 620 can include additional setting options, such as options for selecting universal posture definitions, which may correspond to poses/movements that are performed across a range of devices; options for selecting device specific posture definitions, which correspond to poses/movements that a specific to a particular device; and/or other settings that can be used to define the posture map, as described herein.

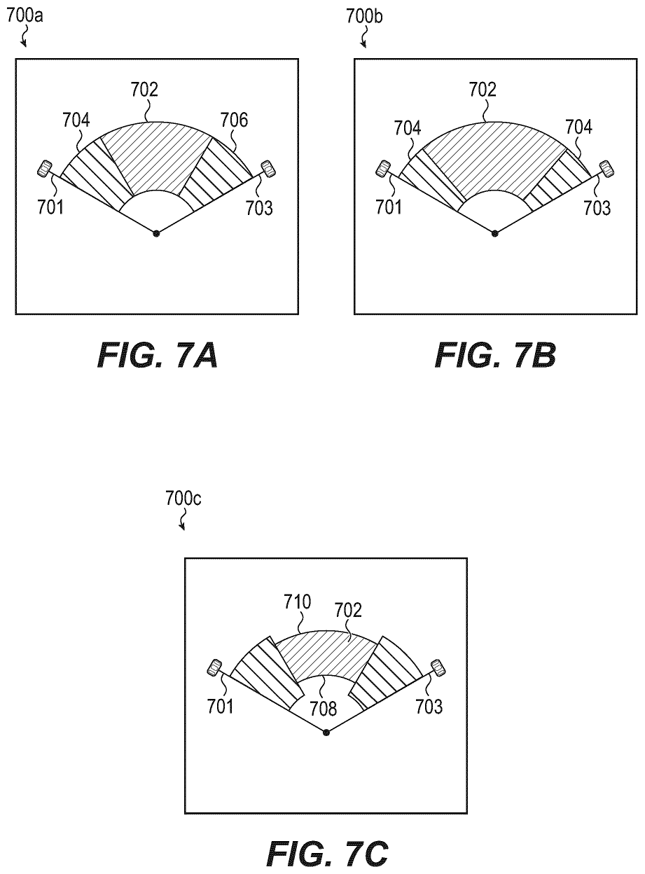

FIGS. 7A-7B show an example user interface 700 for defining boundary regions of a posture map, as described herein. The user interface 700 can be an example of the user interfaces described herein and includes a multi-dimensional plot showing a visual representation of the posture map. In the examples shown in FIGS. 7A-7C the system includes two sensors and the posture map is defined using two component dimensions each corresponding to a different sensor. For example, each user interface 700 can include a first component dimension 701 corresponding to a first sensor, and a second component dimension 703 corresponding to a second sensor.

FIG. 7A shows an example of boundary map that includes a first boundary region 702, a second boundary region 704 and a third boundary region 706. In some cases, each of the boundary regions can be assigned a posture definition as described herein. In other cases, one or more of the boundary regions may be assigned other definitions, which associate a boundary region with an alternative parameter that is not used for posture definition. For example, one or more of the boundary regions can be assigned as a buffer region.

For example, in FIG. 7A, a first instance of the user interface 700a shows an example of the second boundary region 704 and the third boundary region 706 as buffer regions. The buffer regions may be regions of the posture map that are not assigned a posture definition and instead are associated with an alternative function. In some cases, the buffer region(s) can define functionality such as maintaining a current pose or resetting to a default pose. For example, in response to the resultant vector corresponding to the buffer region (e.g., being positioned within the buffer region), the buffer region parameters may cause the system to maintain a current pose. In some cases, if the resultant vector moves from the first boundary region 702 associated with a particular posture definition, into the second boundary region 704, designated first boundary region 702, to the buffer region (second boundary region 704) can cause the system to transition the device to a default pose. The buffer regions can be displayed in the multi-dimensional plot using a buffer object, which may be a distinct visual object designating a particular region as a boundary region.

As described herein, the boundary regions may each include one or more selectable boundaries and the user interface 700 can be configured to allow user inputs to change a location of one or more of the selectable boundaries. For example, FIG. 7B shows a second instance of the user interface 700b in which the area of the first boundary region 702 is expanded in a radial direction to increase the size of the first boundary region 702 and decrease the sizes of the second boundary region 704 and the third boundary region 706. For example, the user interface may allow inputs that select and move (e.g., drag) a boundary edge to change a location of the selected edge within the posture space.

FIG. 7C shows a third instance of the user interface 700c in which a first selectable boundary edge 708 associated with an activation threshold is modified, and a second selectable boundary edge 710 associated with a deactivation threshold is also modified. In some cases, the activation threshold may define a minimum magnitude of a combined resultant vector that is needed to cause a posture definition to be performed. For example, the pose/movement associated with the first boundary region 702 is not performed until the resultant vector moves into the first boundary region and surpasses the first boundary edge 708. Accordingly, moving the first selectable boundary edge 708 further away from the origin (e.g., along a radial direction) increases the activation threshold and requires a greater signal magnitude to activate the associated pose/movement. Moving the first selectable boundary edge 708 closer to the origin (e.g., along a radial direction) decreases the activation threshold and requires lower signal magnitude to active the associated pose/movement.

In response to a change to a location of one or more of the selectable boundary edges, the system can be configured to update the posture map to incorporate the changes, as described herein.

FIGS. 8A-8B show an example user interface for defining boundary wall objects of a posture map, as described herein. The user interface 800 can be an example of the user interfaces described herein and include a multi-dimensional plot showing a visual representation of the posture map. In the examples shown in FIGS. 8A-8B the system includes two sensors and the posture map is defined using two component dimensions each corresponding to a different sensor. For example, each user interface 800 can include a first component dimension 801 corresponding to a first sensor, and a second component dimension 803 corresponding to a second sensor.

The user interface 800 shows a visual example of a posture map that includes a first boundary region 802, which can be associated with a first posture definition, and a second boundary region 804, which can be associated with a second posture definition, as described herein. In some cases, a resultant vector may travel from the first boundary region 802 and into the second boundary region 804, for example, based on changes in detected muscle contractions.

FIG. 8A shows an example of a first instance of the user interface 800a in which the first boundary region 802 and the second boundary region 804 share a common edge 806 and the edge 806 is open. In these examples, and in response to the resultant vector moving from the first boundary region 802 to the second boundary region 804, the system can cause the prosthetic device to transition from a first pose (associated with the first posture definition) and to a second pose (associate with a second posture definition).

In some cases, it may be desirable to prevent transitions from the first posture to the second posture and/or limit the direction of the transition. In these cases, the user interface 800 may allow a user to define boundary walls that are incorporated into the posture map. The boundary walls may be configured to prevent specific movements such as the direct transition from the first pose to the second pose. For example, the user interface 800 can include a boundary wall control 805 that allows a user to activate and/or define locations of boundary walls within the multi-dimensional plot.

FIG. 8B shows an example of a second instance of the user interface 800b with a boundary wall defined between the first boundary region 802 and the second boundary region 804. For example, in response to detecting an input to the boundary wall control 805 and/or designating the common edge as the location for a boundary wall, the user interface can display a boundary wall object 808, which indicates the inclusion of a boundary wall between the first boundary region 802 and the second boundary region 804. In some cases, the boundary wall can be bidirectional and prevent a transition of the resultant vector from the first boundary region 802 to the second boundary region 804 and vice versa. In other cases, the boundary wall can be unidirectional and prevent a transition in one direction (e.g., prevent a transition from the first boundary region 802 to the second boundary region 804), while allowing a transition in another direction (e.g., allow a transition from the second boundary region 804 to the first boundary region 802).

In cases, where a boundary wall prevents a transitions between different boundary regions, the system may maintain a current pose/operation associated with the active boundary region or transition to a default pose/operation in response to the resultant vector intersecting the boundary wall. In some cases, the boundary wall preventing a transition between two different boundary regions may require that the resultant vector enter each of the boundary regions along a specific boundary edge such as an inner boundary edge associated with an activation threshold, as described herein.

In other cases, the boundary wall can define a threshold for allowing a transition between boundary regions. For example, the threshold may be a time threshold that the resultant vector is associated with in another boundary region before allowing the transition and corresponding change in the device pose/movement. As another example, the threshold may be based on a magnitude associated with a transition and the resultant vectors associated with a change in magnitude that satisfies a threshold (e.g., speed, velocity, acceleration of the resultant vector to a different boundary region) are required before allowing the transition between the boundary regions, and corresponding change in the device pose/movement.

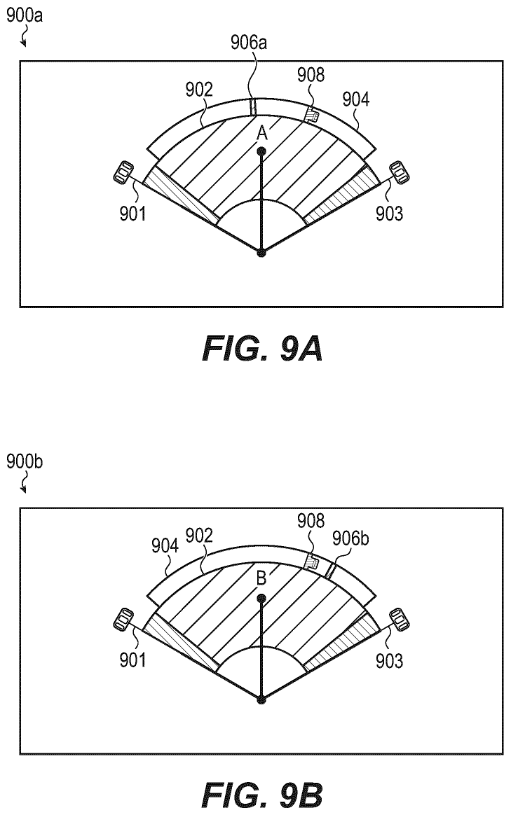

FIGS. 9A-9B show an example user interface 900 for defining secondary correlation of a posture map, as described herein. The user interface 900 can be an example of the user interfaces described herein and includes a multi-dimensional plot showing a visual representation of the posture map. In the examples shown in FIGS. 9A-9B the system includes two sensors and the posture map is defined using two component dimensions each corresponding to a different sensor. For example, each user interface 900 can include a first component dimension 901 corresponding to a first sensor, and a second component dimension 903 corresponding to a second sensor.

The user interface 900 shows a visual example of a posture map that includes a first boundary region 902, which can be associated with a first posture definition. In some cases, the system is configured to determine a scalar quantity of the resultant vector, which may correspond to a speed, acceleration, and/or other parameter determined from detected user signals. The posture map can define a secondary correlation between the scalar quantity and a second posture definition. For example, in response to the scalar quantity (e.g., a speed of the resultant vector entering the boundary region) satisfying a threshold, the system can cause the prosthetic device to perform an operation associated with the second posture definition. In some cases, the second posture definition is related/complimentary to the first posture definition. For example, the second posture definition can define parameters controlling a speed, force, etc., of the first posture definition. In other examples, the second posture definition can be an additional movement.

The user interface 900 can include a multi-dimensional plot, as described herein, which can display a visual object 904 defining a threshold 908 for activating a second posture definition. In some cases the user interface can include a dynamic graphical icon 906 within the visual object 904 and indicating a value of the determined scalar quantity with respect to the threshold 908. FIGS. 9A-9B show an example of the dynamic graphical icon 906 as a bar that moves along the visual object 904. FIG. 9A shows a first instance of the user interface 900a illustrating an example of the dynamic graphical icon 906a indicating a value of the scalar quantity below the threshold 908, and accordingly the system does not perform the second posture definition. FIG. 9B shows a second instance of the user interface 900b illustrating an example of the dynamic graphical icon 906b that indicates a value of the scalar quantity above the threshold, thereby causing the system to perform the second posture definition.

The user interface 900 can be configured to allow a user to assign different functions (e.g., posture definitions, secondary movements, etc.) to the second posture definition, and/or adjust parameters of the second posture definition such as adjusting the threshold 908. For example, a user can slide the threshold 908 icon to increase or decrease a threshold associated with the secondary function.

FIGS. 10A-10B show an example user interface 1000 for adding additional boundary regions to a posture map, as described herein. The user interface 1000 can be an example of the user interfaces described herein and includes a multi-dimensional plot showing a visual representation of the posture map. In the examples shown in FIGS. 10A-10B the system includes two sensors and the posture map is defined using two component dimensions each corresponding to a different sensor. For example, each user interface 1000 can include a first component dimension 1001 corresponding to a first sensor, and a second component dimension 1003 corresponding to a second sensor.

The user interface 1000 shows a visual example of a posture map that includes a first boundary region 1002, which can be associated with a first posture definition, and a second boundary region 1004 which can be associated with a second posture definition. In some cases, the user interface 1000 can include an option 1008 to add an additional boundary region to the posture map. Selection of the option 1008 can cause an additional boundary region to be added to the interface 1000 and update the posture map accordingly. FIG. 10A shows a first instance of the user interface 1000a with two boundary regions 1002, 1004, prior to adding an additional boundary region. FIG. 10B shows a second instance of the user interface 1000b with three boundary regions 1002, 1004 and an added third boundary region 1006. Additional boundary regions can be added using a first graphical option 1008a or a second graphical option 1008b displayed in the user interface 1000.

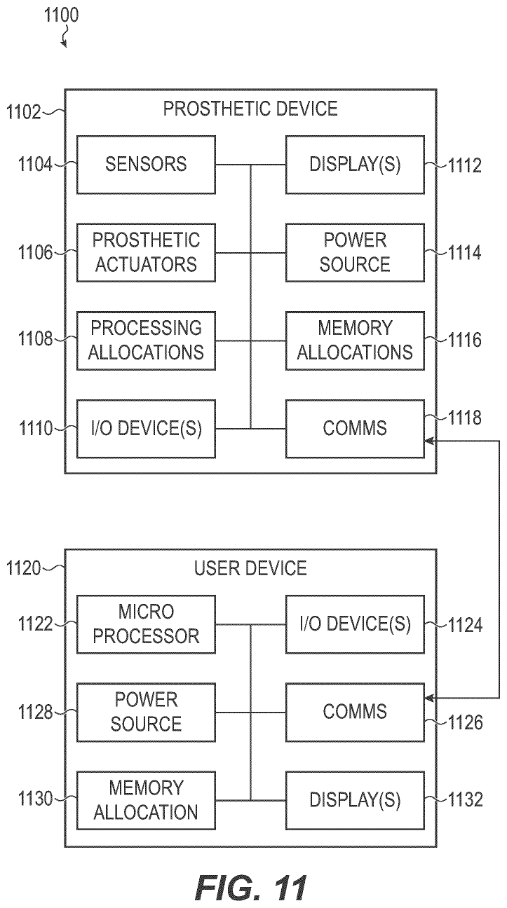

FIG. 11 shows an example electrical block diagram of a prosthetic system 1100 that may perform the operations described herein. The prosthetic system 1100 can have a prosthetic device 1102, which can be an example of the prosthetic devices described herein. The prosthetic device 1102 can include one or more sensors 1104, one or more prosthetic actuators 1106, processing allocations 1108, input/output (I/O) devices 1110, a display 1112 (which may, in some examples, be optional like other components shown in FIG. 11), a power source 1114, memory allocation 1116, and communication devices (COMMS) 1118. The prosthetic system 1100 can also include a user device 1120 that is an example of the user devices described herein. The user device 1120 can include a micro-processor 1122, I/O devices 1124, communication devices (COMMS) 1126, a power source 1128, memory allocation 1130, and a display 1132.

The prosthetic system may also include one or more sensors 1104 positioned at different locations on the prosthetic system 1100. The sensor(s) 1104 can be myoelectric sensors or other suitable sensors (e.g., sonomyographic sensors) that are configured to sense volitional motor control signals (e.g., myoelectric signals, sonomyography signals, neural signals, and/or other biological signals) in a residual limb (or other locations) of a user as described herein. Additionally, the system can include other sensors 1104 configured to sense one or more types of parameters, such as but not limited to, pressure, light, touch, heat, movement, relative motion, biometric data (e.g., biological parameters), and so on.

For example, these additional sensor(s) 1104 may include a thermal sensor, a position sensor, a light or optical sensor, an accelerometer, a pressure transducer, a gyroscope, a magnetometer, a health monitoring sensor, and so on. Additionally, the one or more sensors 1104 can utilize any suitable sensing technology, including, but not limited to, capacitive, ultrasonic, resistive, optical, ultrasound, piezoelectric, and thermal sensing or imaging technology.

The prosthetic actuators 1106 can include motors, hydraulic actuators, mechanical linkages, gear driven systems, and/or other electromechanical systems that create movement in one or more portions of the prosthetic device such as a prosthetic hand. The prosthetic actuators can include motors and assemblies that drive movement of one or more prosthetic fingers, wrist movement, other hand movement, and/or movements of other joints such as an elbow in the case of an upper arm amputation.

The processing allocations 1108 can be implemented as any electronic device capable of processing, receiving, or transmitting data or instructions. For example, the processing allocations 1108 can be a microprocessor, a central processing unit (CPU), an application-specific integrated circuit (ASIC), a digital signal processor (DSP), or combinations of such devices. As described herein, the term “processor” is meant to encompass a single processor or processing unit, multiple processors, multiple processing units, or other suitable computing element or elements. The processing unit can be programmed to perform the various aspects of the systems described herein.

It should be noted that the components of the prosthetic system 1100 can be controlled by multiple processors. For example, select components of the prosthetic system 1100 (e.g., a sensor 1104) may be controlled by a first processor and other components of the prosthetic system 1100 (e.g., the prosthetic actuators 1106) may be controlled by a second processor, where the first and second processors may or may not be in communication with each other.

The I/O devices 1110 can be any suitable mechanisms that allow a user to provide input to the prosthetic device 1102 and/or the user device 1120 and receive feedback from these devices. In some cases, the I/O devices 1110 can be touch- and/or force-sensitive and include or be associated with touch sensors and/or force sensors that extend along the output region of the display and which may use any suitable sensing elements and/or sensing techniques.

Using touch sensors, the prosthetic device 1102 and/or user device 1120 may detect touch inputs applied to a display region including detecting locations of touch inputs, motions of touch inputs (e.g., the speed, direction, or other parameters of a gesture applied to the display), or the like. Using force sensors, the prosthetic device 1102 and/or user device 1120 may detect amounts or magnitudes of force associated with touch events applied to the display. The touch and/or force sensors may detect various types of user inputs to control or modify the operation of the device, including taps, swipes, multiple finger inputs, single- or multiple-finger touch gestures, presses, and the like.

Additionally or alternatively, the I/O devices 1110 can include buttons, or other suitable input devices. In some cases, the I/O devices 1110 can include a microphone and/or speaker and be configured to output sounds and receive voice-feedback. For example, a user may be able to provide voice feedback via the user device 1120 on movement performed by their prosthetic device.

As noted above, the prosthetic system 1100 may optionally include the display 1112 such as a liquid-crystal display (LCD), an organic light emitting diode (OLED) display, a light emitting diode (LED) display, or the like. If the display 1112 is an LCD, the display 1112 may also include a backlight component that can be controlled to provide variable levels of display brightness. If the display 1112 is an OLED or LED type display, the brightness of the display 1112 may be controlled by modifying the electrical signals that are provided to display elements. The display 1112 may correspond to any of the displays shown or described herein.

The power source 1114 can be implemented with any device capable of providing energy to the prosthetic device 1102. For example, the power source 1114 may be one or more batteries or rechargeable batteries. Additionally or alternatively, the power source 1114 can be a power connector or power cord that connects the prosthetic device 1102 to another power source, such as a wall outlet.

The memory allocations 1116 can store electronic data that can be used by the prosthetic device 1102. For example, the memory 1116 can store electrical data or content such as, for example, audio and video files, documents and applications, device settings and user preferences, timing signals, control signals, and data structures or databases. The memory 1116 can be configured as any type of memory. By way of example only, the memory 1116 can be implemented as random access memory, read-only memory, Flash memory, removable memory, other types of storage elements, or combinations of such devices.

The communication device 1118 can transmit and/or receive data from a user or another electronic device. A communication device can transmit electronic signals via a communications network, such as a wireless and/or wired network connection. Examples of wireless and wired network connections include, but are not limited to, cellular, Wi-Fi, Bluetooth, IR, and Ethernet connections. In some cases, the communication device 1118 can communicate with an external electronic device, such as a smartphone, electronic device, or other portable electronic device, as described here.

The user device 1120 can include a micro-processor 1122, I/O devices 1124, communication devices 1126, a power source 1128, memory allocations 1130, and a display 1132, which can be housed in an independent structure that functions independently of the prosthetic device 1102.

In other cases, the user device 1120 can be configured to conductively couple to the prosthetic device 1102, for example by a data and/or power cable.

The user device 1120 may be any suitable electronic device including, but not limited to a cellular phone, smart watch, tablet device, laptop computer, wearable device, and so on. These examples are not limiting; the user device 1120 can be any suitable electronic device.

The micro-processor 1122 of the user device 1120 can be configured to cooperate with the memory allocations 1130 to instantiate one or more instances of software configured to perform, coordinate, or supervise one or more operations described herein. For example, in some embodiments, the user device 1120 can be configured to instantiate controller software configured to execute one or more classifications or retraining operations, such as described above.

In other cases, the user device 1120 can be configured to instantiate an instance of software that records a timeline of events/actions performed by the prosthetic device 1102. In these examples, the user can operate the user device 1120 to select an action performed by the prosthetic at a known time, but not immediately in the past. For example, in some circumstances, the user may not be immediately able to signal to the prosthetic device that a particular action or pose was incorrect. In these examples, the user may leverage the user device 1120 to review actions (which may be tagged with time and/or geolocation) taken by the prosthetic, so that the user can provide input indicating that one or more actions were not correct. In simpler phrasing, in some embodiments, the user device 1120 can be used to correct any historical action taken by the prosthetic device, not just actions taken immediately preceding or near-in-time with the user's input.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A prosthetic system comprising:
a first sensor configured to detect first signals at a first location of a user;
a second sensor configured to detect second signals at a second location of the user; and
an electronic device configured to:
receive first sensor data based on the first signals detected by the first sensor;
receive second sensor data based on the second signals detected by the second sensor; and
determine a resultant vector using the first sensor data and the second sensor data;
display a user interface comprising a multi-dimensional plot corresponding to a posture map, the posture map defining an association between one or more boundary regions and one or more posture definitions used to control movement of a prosthetic device, the multi-dimensional plot comprising:
a first visual object defining a first boundary region of the one or more boundary regions within the multi-dimensional plot and corresponding to a first posture definition of the posture map; and
a second visual object defining a second boundary region of the one or more boundary regions within the multi-dimensional plot and corresponding to a second posture definition of the posture map;
display a dynamic graphical icon on the multi-dimensional plot indicating a current correlation of the resultant vector with respect to the one or more boundary regions of the posture map;
receive a user input changing a location of at least a portion of the first boundary region within the multi-dimensional plot; and
update the posture map based on the change to the location of the portion of the first boundary region.

2. The prosthetic system of claim 1, wherein:
the first visual object comprises a first selectable edge corresponding to an activation threshold for the first posture definition;
the user interface is configured to allow user inputs to change a location of the first selectable edge within the multi-dimensional plot; and
the electronic device is configured to update the posture map in response to a change in the location of the first selectable edge.

3. The prosthetic system of claim 1, wherein:
the first visual object comprises a second selectable edge corresponding to an activation boundary for the first posture definition;
the user interface is configured to allow user inputs to change a location of the second selectable edge within the multi-dimensional plot; and
the electronic device is configured to update the posture map in response to a change in the location of the second selectable edge.

4. The prosthetic system of claim 1, wherein:
the electronic device is further configured to determine a scalar quantity of the resultant vector;
the posture map further defines a secondary correlation between the scalar quantity and a third posture definition; and
the multi-dimensional plot comprises a third visual object defining a threshold for activating the third posture definition.

5. The prosthetic system of claim 4, wherein the electronic device is configured to display a second dynamic graphical icon within the third visual object and indicating a value of the determined scalar quantity with respect to the threshold.

6. The prosthetic system of claim 4, wherein:
the user interface is configured to allow user inputs to change a location of the threshold within the multi-dimensional plot; and
the electronic device is configured to update the posture map in response to a change in the location of the threshold.

7. The prosthetic system of claim 1, wherein:
the posture map comprises a buffer region adjacent to at least one of the one or more boundary regions;
the buffer region defines areas of the posture map that do not correspond to a posture definition of the one or more posture definitions; and
the multi-dimensional plot comprises a buffer object displaying a location of the buffer region with respect to the one or more boundary regions.

8. The prosthetic system of claim 7, wherein:
the buffer object comprises one or more selectable boundaries;
the user interface is configured to allow user inputs to change a location of the one or more selectable boundaries; and
the electronic device is configured to update the posture map in response to a change in the location of the one or more selectable boundaries.

9. The prosthetic system of claim 1, wherein the electronic device is configured to:
in response to selection of a boundary region of the one or more boundary regions, cause display in the user interface of a selection object comprising multiple posture objects, each of the multiple posture object corresponding to a different posture definition of the one or more posture definitions; and
in response to selection of a particular posture object of the multiple posture objects:
assign a corresponding posture definition to the selected boundary region; and
update the posture map to correlate the selected boundary region with the corresponding posture definition.

10. The prosthetic system of claim 1, wherein:
the user interface comprises a boundary wall object configured to define a boundary between the first boundary region and the second boundary region; and
the boundary is configured to prevent a transition between the first posture definition and the second posture definition.

11. A method of operating a prosthetic device, the method comprising:
receiving first sensor data based on first myoelectric signals detected by a first sensor;
receiving second sensor data based on second myoelectric signals detected by a second sensor;
determining a resultant vector using the first sensor data and the second sensor data;
causing display of a user interface comprising a multi-dimensional plot corresponding to a posture map, the posture map defining an association between one or more boundary regions and one or more posture definitions used to control movement of the prosthetic device,
causing display of a dynamic graphical icon on the multi-dimensional plot indicating a current correlation of the resultant vector with respect to the one or more boundary regions of the posture map;
receiving a user input changing a location of at least a portion of a first boundary region within the multi-dimensional plot; and
updating the posture map based on the change to the location of the portion of the first boundary region.

12. The method of claim 11, wherein:
the multi-dimensional plot comprises:
a first visual object defining the first boundary region of the one or more boundary regions within the multi-dimensional plot and corresponding to a first posture definition of the posture map; and
second visual object defining a second boundary region of the one or more boundary regions within the multi-dimensional plot and corresponding to a second posture definition of the posture map.

13. The method of claim 12, further comprising, in response to selection of a boundary region of the one or more boundary regions, causing display in the user interface of a selection object comprising multiple posture objects, each of the multiple posture object corresponding to a different posture definition of the one or more posture definitions.

14. The method of claim 13, further comprising:
in response to selection of a particular posture object of the multiple posture objects:
assigning a corresponding posture definition to the selected boundary region; and
updating the posture map to correlate the selected boundary region with the corresponding posture definition.

15. A prosthetic device comprising:
a first sensor configured to detect myoelectric signals at a first location of a user;
a second sensor configured to detect myoelectric signals at a second location of the user;
memory storing a posture map defining a correlation between one or more boundary regions defined by first and second component dimensions and one or more posture definitions used to control movement of the prosthetic device, the one or more boundary regions comprising:
a first boundary region defining a first posture definition;
a second boundary region defining a second posture definition; and
a buffer region separating the first boundary region and the second boundary region and defining a region of the posture map that does not correspond to a posture definition; and
a controller configured to:
receive first sensor data based on the myoelectric signals detected by the first sensor;
receive second sensor data based on the myoelectric signals detected by the second sensor; and
determine a resultant vector using the first sensor data and the second sensor data;
determine an intended posture definition based on mapping the resultant vector with respect to the posture map; and
cause the prosthetic device to perform one or more operations associated with the intended posture definition.

16. The prosthetic device of claim 15, wherein, in response to determining that the mapping of the resultant vector corresponds to the buffer region, the controller is configured to maintain a current posture of the prosthetic device.

17. The prosthetic device of claim 15, wherein:
the resultant vector is a first instance of the resultant vector corresponding to sensor signals received at a first time;
the controller is configured to:
receive third sensor data based on the myoelectric signals detected by the first sensor at a second time;
receive fourth sensor data based on the myoelectric signals detected by the second sensor at the second time; and
determine a second instance of the resultant vector.

18. The prosthetic device of claim 17, wherein:
in response to determining that the mapping of the first instance of the resultant vector corresponds to the first boundary region, the controller is configured to cause the prosthetic device to perform a first posture corresponding to the first posture definition; and
in response to determining that the mapping of the second instance of the resultant vector corresponds to the buffer region, the controller is configured to cause the prosthetic device to maintain the first posture.

19. The prosthetic device of claim 15, wherein the posture map defines a boundary wall between the first boundary region and the second boundary region.

20. The prosthetic device of claim 19, wherein, in response to determining that the mapping of the resultant vector corresponds to a transition from the first boundary region to the second boundary region and the posture map defines the boundary wall between the first boundary region and the second boundary region, the controller is configured to cause the prosthetic device to maintain the intended posture.
